(19) United States
(12) Patent Application Publication
(10) Pub. No.: US 2026/0240934 A1
(43) Pub. Date: Aug. 20, 2026

(54) NATURAL MEDICINAL COMPOSITION

(71) Applicant: Sardar M. Jakaria, Cuyahoga Falls, OH (US)

(72) Inventor: Sardar M. Jakaria, Cuyahoga Falls, OH (US)

(21) Appl. No.: 19/531,971

(22) Filed: Feb. 6, 2026

Related U.S. Application Data
(60) Provisional application No. 63/759,576, filed on Feb. 18, 2025.

(51) Int. Cl. A61K 36/71; A61K 9/00; A61K 9/20; A61K 31/122; A61K 31/4453; A61K 36/23; A61K 36/67; A61K 36/9066; A61K 47/22

(52) U.S. Cl. CPC A61K 36/71; A61K 9/0014; A61K 9/0095; A61K 9/20; A61K 31/122; A61K 31/4453; A61K 36/23; A61K 36/67; A61K 36/9066; A61K 47/22

(57) ABSTRACT

A multifunctional all-natural herbal composition is disclosed comprising synergistic combinations of plant-derived bioactive components formulated for oral, topical, or transdermal administration. The composition comprises a bioactive modulation system of one or more botanicals selected from the group of Nigella sativa (i.e. provided as a seed powder, extract, or oil) optionally standardized to thymoquinone; Piper nigrum extract optionally standardized to piperine; and, optionally combined with additional plant-derived components to support immune balance, metabolic regulation, cardiovascular function, musculoskeletal comfort, epithelial integrity, and respiratory support. The composition can further comprise functional botanical modules, lipid carriers, and natural excipients configured to enhance bioavailability, stability, tissue residence time, and/or sensory performance. Representative delivery embodiments are configurable as capsules, tablets, syrups, oils, balms, creams, gels, topical ointments, and related dosage forms. The disclosed compositions define a unified and modular botanical platform suitable for nutraceutical, cosmeceutical, and pharmaceutical-grade development.

### CROSS REFERENCE TO RELATED APPLICATION

The present application claims priority to, and the benefit of, U.S. Provisional Application No. 63/759,576, which was filed on Feb. 18, 2025 and is incorporated herein by reference in its entirety.

### FIELD OF THE INVENTION

The present invention generally relates to the field of natural and botanical compositions. More particularly, the present invention relates to multifunctional, all-natural bioactive compositions comprising synergistic combinations of plant-derived bioactive components configured for oral, topical, or cosmetic administration. The disclosed compositions are designed to support, modulate, or enhance physiological functions through coordinated botanical interactions rather than single-target pharmacological intervention.

In exemplary embodiments, the compositions comprise one or more primary botanical actives selected from Nigella sativa provided in seed powder, extract, or oil form, optionally standardized to thymoquinone, and Piper nigrum extract optionally standardized to piperine to enhance bioavailability and functional interaction of co-administered botanicals. The primary bioactive system may be optionally combined with additional plant-derived components selected to support immune balance, metabolic regulation, cardiovascular function, musculoskeletal comfort, skin and hair health, digestive support, or respiratory comfort.

The compositions may further include lipid carriers, functional botanical modules, and natural excipients or stabilizers configured to improve delivery, stability, sensory performance, or tissue residence time. Representative embodiments may be formulated as capsules, tablets, syrups, oils, balms, creams, gels, or related dosage forms, as illustrated in Tables 1-20 and FIGS. 1-3. Accordingly, the present disclosure provides a unified, modular botanical platform adaptable across a range of nutraceutical, cosmeceutical, and related applications.

The present invention relates to multifunctional herbal compositions comprising one or more botanically derived actives, extracts, essential oils, or plant-based powders formulated alone or in combination with nutraceutically, cosmetically, or pharmaceutically acceptable excipients. The disclosed compositions are configured to provide coordinated biological effects through anti-inflammatory, antioxidant, antimicrobial, immunomodulatory, soothing, or restorative botanical interactions rather than single-target pharmacological suppression.

The invention encompasses a range of dosage forms including, without limitation, capsules, tablets, granules, powders, suspensions, syrups, ointments, balms, creams, gels, lotions, serums, or oils, adapted primarily for oral, topical, or transdermal administration. The specific dosage form and excipient system may be selected based on the intended use, delivery route, and performance characteristics while maintaining the functional role of the active botanical constituents.

Unless otherwise stated, each herbal or natural ingredient disclosed herein may be present individually or in combination within the composition in an effective amount within the ranges disclosed in the specification and representative tables. The relative ratios of active components and excipients may be adjusted according to dosage strength, formulation type, or delivery system, provided that the core functional interaction and system architecture of the composition are maintained.

This disclosure is intended to encompass equivalent formulations, partial combinations, sub-compositions, and derivatives thereof that operate within the same functional framework. Compositions comprising the disclosed botanical actives in forms including raw plant material, standardized extract, isolated phytochemical, encapsulated, nano-dispersed, or liposomal embodiments are contemplated, provided such compositions are consistent with the structural and functional features set forth in the claims.

Herbal compositions described herein for capsule dosage forms may equivalently be formulated into compressed tablets, granules, or orally dispersible solids utilizing substantially the same active components in comparable proportions. Differences among such solid dosage forms reside primarily in the selection of binders, matrix formers, and excipients used to facilitate granulation, compaction, disintegration, or release performance.

Suitable binders and matrix formers include, by way of example, microcrystalline cellulose, acacia gum, guar gum, maltodextrin, starch-based materials, polyvinylpyrrolidone, hydroxypropyl cellulose, and hydroxypropyl methylcellulose, which may be used individually or in combination. Lubricants, glidants, and disintegrants such as magnesium stearate, colloidal silica, and croscarmellose sodium may likewise be incorporated as appropriate to support manufacturability, flow, and dissolution characteristics.

Accordingly, the present invention encompasses compositions, dosage forms, and delivery systems employing the disclosed botanical actives and excipients that achieve substantially similar functional outcomes through equivalent structural and formulation architectures, as defined by the appended claims.

### BACKGROUND

In recent years, there has been increasing interest in botanical, nutraceutical, and complementary compositions intended to support physiological balance across a range of health-related conditions. Individuals experiencing conditions associated with inflammation, metabolic imbalance, cardiovascular stress, digestive discomfort, musculoskeletal strain, dermatological irritation, respiratory congestion, microbial imbalance, or general functional decline increasingly seek natural bioactive compositions that are well-tolerated, accessible, and suitable for repeated or long-term use.

Many conventional pharmaceutical interventions, while effective for targeted indications, may be associated with adverse effects, limited tolerability, or high long-term cost. In addition, pharmaceutical therapies are often designed to act on isolated molecular targets and may be less suitable for conditions involving multiple interacting biological pathways. As a result, there exists a continued interest in botanical systems capable of addressing complex physiological conditions through coordinated, multi-pathway interactions.

Existing herbal and botanical products, however, frequently suffer from shortcomings including inconsistent composition, variable bioavailability, and lack of reproducibility. Differences in plant sourcing, extraction methods, excipient selection, and formulation architecture can result in unpredictable performance. Moreover, many commercially available products rely on isolated extracts or single actives without consideration of synergistic botanical matrices, delivery systems, or stabilizing frameworks that influence functional interaction, stability, and tissue exposure.

Accordingly, there exists a long-felt need in the art for a multifunctional botanical composition that provides a reproducible, modular, and scalable framework capable of supporting a range of physiological functions using plant-derived components. There is further a need for such compositions to be adaptable across multiple dosage forms, including oral and topical formats, while maintaining consistent functional performance and stability without reliance on harsh synthetic chemicals or petrochemical carriers.

The present invention addresses these needs by providing a unified botanical composition platform comprising coordinated combinations of plant-derived bioactive components, lipid systems, and natural excipients configured to enhance stability, bioavailability, and functional interaction. Representative embodiments are disclosed in Tables 1-20 and FIGS. 1-3, illustrating modular compositions adaptable for oral, topical, or cosmetic use. The disclosed framework enables consistent formulation across a variety of applications while preserving flexibility for customization based on intended use, delivery route, and performance objectives.

### SUMMARY OF THE INVENTION

The following presents a simplified summary of certain aspects of the disclosed invention in order to provide a basic understanding of the general principles thereof. This summary is not intended to identify key or critical elements of the invention, nor to limit the scope of the claims. The invention is defined solely by the appended claims.

In one embodiment, the disclosed subject matter comprises a multifunctional, all-natural botanical composition configured as a modular system of plant-derived bioactive components. The composition includes a primary bioactive modulation layer comprising Nigella sativa provided in seed powder, extract, or oil form, optionally standardized to thymoquinone, and Piper nigrum extract optionally standardized to piperine to enhance bioavailability and functional interaction. The primary layer may be combined with one or more functional botanical modules selected to support immune balance, metabolic regulation, cardiovascular function, musculoskeletal comfort, digestive support, skin and hair health, respiratory comfort, or related physiological functions.

The compositions may further include lipid carriers, delivery enhancers, antioxidants, and natural excipients configured to improve stability, tissue residence time, sensory performance, or release characteristics. The disclosed system is adaptable to a variety of dosage forms, including but not limited to capsules, tablets, powders, syrups, oils, balms, creams, gels, lotions, or related oral or topical formulations.

In representative embodiments, the composition may be configured as higher-potency or lower-potency formulations by adjusting the relative amounts of the primary bioactive layer and selected functional modules within the ranges disclosed in the specification. Such embodiments may be tailored for different user sensitivities, delivery routes, or intended use cases while maintaining the same core system architecture.

Topical embodiments of the invention comprise lipid-based formulations incorporating the primary bioactive modulation layer in combination with botanical oils, waxes, and soothing or restorative plant-derived components. These embodiments may be configured to support skin barrier integrity, musculoskeletal comfort, neurosensory modulation, or cosmetic performance.

Representative embodiments of the invention are illustrated in Tables 1-20 and FIGS. 1-3, which describe exemplary modular compositions and dosage forms. The disclosed framework provides a unified botanical platform enabling reproducible formulation across multiple applications while preserving flexibility for substitution, customization, and optimization within the scope of the claims.

Numerous advantages and features of the disclosed compositions will become apparent to those skilled in the art upon review of the following detailed description and drawings.

### BRIEF DESCRIPTION OF THE DRAWINGS

The description refers to the accompanying drawings in which like reference numerals refer to like elements throughout the several views, and in which:

FIG. 1 illustrates a block diagram depicting representative botanical components of a multifunctional, all-natural bioactive composition of the present invention, in accordance with one embodiment thereof.

FIG. 2 illustrates a block diagram depicting delivery, stability, and excipient components of the multifunctional all-natural bioactive composition of the present invention, in accordance with one embodiment thereof.

FIG. 3 illustrates a perspective view showing topical application of the multifunctional all-natural bioactive composition to a subject, in accordance with one embodiment of the present invention.

### DETAILED DESCRIPTION OF THE PRESENT INVENTION

The present invention is described with reference to the accompanying drawings, in which like reference numerals refer to like elements throughout. The following detailed description is provided to illustrate representative embodiments of the invention and is not intended to limit the scope of the invention as defined by the appended claims. It will be appreciated by those skilled in the art that various modifications, substitutions, and combinations may be made without departing from the spirit and scope of the invention.

As discussed above, there exists a need for botanical composition systems capable of supporting multiple physiological functions through coordinated, multi-pathway interactions. Such systems are particularly desirable where conditions are characterized by inflammation, metabolic imbalance, oxidative stress, microbial imbalance, barrier dysfunction, or neurosensory irritation, and where long-term tolerability and formulation flexibility are important.

In one representative embodiment, the present invention comprises a multifunctional botanical composition configured as a modular system of plant-derived bioactive components. The composition includes a primary bioactive modulation layer comprising Nigella sativa provided in seed powder, extract, or oil form, optionally standardized to thymoquinone, and Piper nigrum extract optionally standardized to piperine. The primary layer may be combined with one or more functional botanical support modules, selected according to the intended use, delivery route, or performance objective.

The functional botanical support modules may include, by way of example and not limitation, botanicals associated with anti-inflammatory support, antioxidant balance, metabolic regulation, digestive comfort, musculoskeletal support, immune balance, skin and hair maintenance, respiratory comfort, or neurosensory modulation. Representative botanical components suitable for inclusion in one or more modules are described throughout the specification and illustrated in Tables 1-20.

The composition may further comprise lipid carriers, botanical oils, waxes, antioxidants, and natural excipients configured to enhance bioavailability, stability, tissue residence time, or sensory performance. Each botanical component may be present individually or in combination in effective amounts within the ranges disclosed in the specification, and the relative ratios of components may be adjusted while maintaining the functional role and system architecture of the composition.

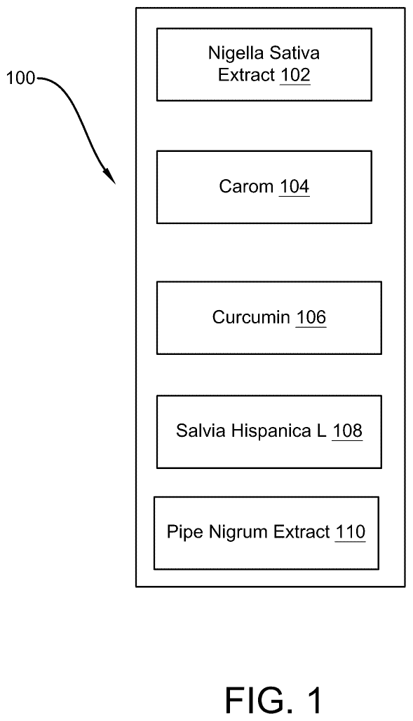

Referring to FIG. 1, a representative block diagram of the multifunctional botanical composition 100 is shown. In this embodiment, the composition includes Nigella sativa 102, Carom (Trachyspermum ammi) 104, Curcuma longa extract 106, Salvia hispanica L. 108, and Piper nigrum extract 110. These components are arranged to provide coordinated botanical interaction rather than isolated single-target activity.

Nigella sativa 102 is a principal botanical component of the invention and is associated with antioxidant, soothing, and barrier-supportive properties. When incorporated into the disclosed system, Nigella sativa contributes to systemic and topical formulations intended to support immune balance, inflammatory modulation, skin barrier integrity, and general physiological resilience.

Carom (Trachyspermum ammi) 104 is optionally included as a botanical component associated with digestive comfort, respiratory support, and microbial balance. Carom may be provided as a seed powder, extract, or oil and may be incorporated into oral or topical embodiments.

Curcuma longa extract 106 is optionally included as a botanical component associated with antioxidant balance and inflammatory modulation. In representative embodiments, Curcuma longa extract may be combined with Piper nigrum extract to enhance functional interaction and bioavailability.

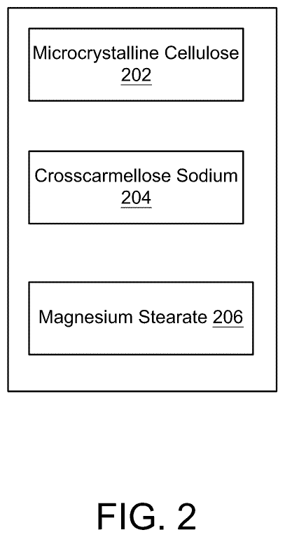

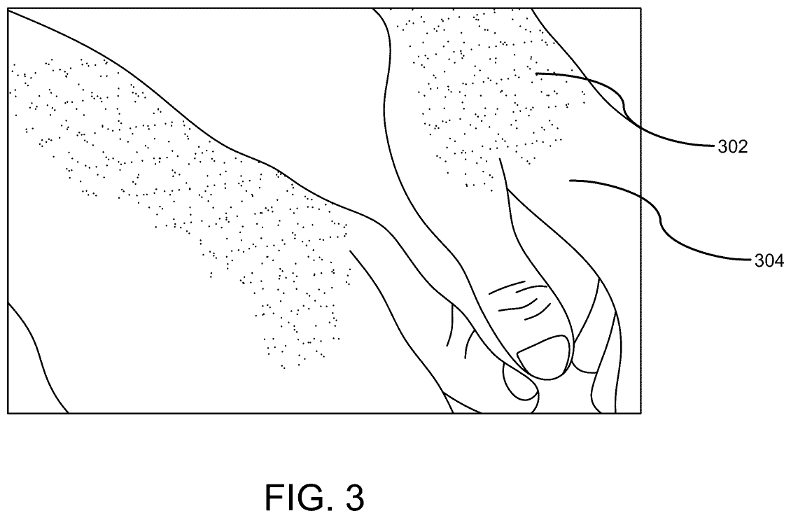

Salvia hispanica L. 108 and Piper nigrum extract 110 may further contribute to lipid balance, delivery efficiency, and functional interaction among botanical components. Piper nigrum extract, in particular, may enhance absorption or functional persistence of co-administered botanicals. The combined system thereby provides a flexible, reproducible botanical platform adaptable across oral and topical embodiments, as further illustrated in Tables 1-20 and FIGS. 1-3.

Table 1 shows approximate and exemplary concentrations of various ingredients in a representative core multi-layer oral composition of the multifunctional all-natural medicinal or pharmaceutical composition 100. The composition of Table 1 is configured as a modular system suitable for promoting immune health and for providing advanced natural support across a plurality of physiological conditions, including inflammatory, immune, metabolic, neuro-endocrine, and circulatory support.

In the representative embodiment, Nigella sativa, provided as a seed powder, extract, or oil and standardized to approximately 0.1-5 percent thymoquinone, serves as a primary immunomodulatory and antioxidant constituent. Piper nigrum extract, standardized to approximately 90-95 percent piperine, is included to enhance oral bioavailability of thymoquinone and other co-administered botanical components through modulation of intestinal metabolic enzymes and transport pathways.

The composition further comprises one or more functional support modules, selected individually or in combination depending on the intended physiological support. Such modules may include, by way of example and not limitation, anti-inflammatory and immune support botanicals, digestive and metabolic support botanicals, neuro-endocrine and stress-modulating botanicals, cardiovascular and circulatory support botanicals, and hormonal or reproductive support botanicals, each present within the exemplary ranges set forth in Table 1.

Additional delivery, stability, and excipient components may be included, such as natural binders, fillers, and antioxidants, to provide a stable oral dosage form suitable for capsules, tablets, or powders, without reliance on synthetic binders or preservatives.

The composition of Table 1 represents a core embodiment of the invention. Tables 2-20 illustrate exemplary variations in which one or more functional support modules are selected, substituted, added, or omitted, or wherein relative amounts are adjusted within the disclosed ranges, without altering the core system architecture defined by the primary bioactive modulation layer and delivery interaction layer.

The compositions set forth in Tables 1-20 are representative, non-limiting embodiments illustrating functional ingredient classes, modular combinations, and concentration ranges. Specific commercial or clinical formulations may employ fixed ratios or narrower ranges derived from these embodiments without departing from the scope of the invention.

TABLE 1
Immune Health & Cancer Support (Oral Capsule/Tablet)

| | | Exemplary | |
|---|---|---|---|
| | | Amount (per unit | |
| Component Category | Ingredient | dose) | |
| A. Primary Bioactive Modulation | Nigella sativa (seed powder, | 25-300 | mg |
| Layer | extract, or oil; standardized to 0.1- | | |
| | 5% thymoquinone) | | |
| | Piper nigrum extract (standardized | 2-20 | mg |
| | to 90-95% piperine) | | |
| B. Functional Support Modules (one | | | |
| or more selected depending on | | | |
| intended physiological support) | | | |
| B1. Anti-Inflammatory/Immune | Curcuma longa extract | 5-150 | mg |
| Module | (curcuminoids) | | |
| | Quercetin | 5-100 | mg |
| | Resveratrol | 5-50 | mg |
| | Green Tea extract (catechins/ | 5-200 | mg |
| | EGCG) | | |
| B2. Digestive/Metabolic Module | Carom (Trachyspermum ammi) | 5-200 | mg |
| | powder or extract | | |
| | Triphala extract | 10-150 | mg |
| | Ginger powder or extract | 5-100 | mg |
| | Cinnamon bark extract | 5-120 | mg |
| | Berberine extract | 50-150 | mg |
| B3. Neuro-Endocrine/Stress Module | Ashwagandha extract | 25-150 | mg |
| | (withanolides) | | |
| | Panax ginseng extract | 25-150 | mg |
| | Bacopa monnieri or Lion's Mane | 25-200 | mg |
| | extract (optional) | | |
| B4. Cardiovascular/Circulatory | Hawthorn berry extract | 25-150 | mg |
| Module | Ginkgo biloba extract | 25-120 | mg |
| | Danshen (Salvia miltiorrhiza) | 25-150 | mg |
| | extract | | |
| | L-Citrulline or Nattokinase | 25-200 | mg |
| B5. Hormonal/Reproductive Module | Shatavari root extract | 25-150 | mg |
| | Vitex agnus-castus extract | 25-150 | mg |
| | Maca root powder or extract | 25-150 | mg |
| C. Delivery, Stability, and Excipient | Rice flour, acacia gum, MCC, or | q.s. | |
| Layer | combinations thereof | | |
| | Rosemary extract or Vitamin E | 0.1-20 | mg |
| | (natural antioxidants) | | |
| Total Unit Dose | | 400-700 | mg |

The composition of Table 1 represents a core embodiment of the invention. One or more functional support modules (B1-B5) may be selected, substituted, added, or omitted depending on the intended physiological support without altering the core system architecture defined by the primary bioactive modulation layer and delivery interaction layer. One or more botanical components may be selected, substituted, combined, or fixed at specific ratios within the disclosed ranges without altering the core system architecture or departing from the scope of the claimed invention.

Table 2 illustrates exemplary cardiovascular and circulatory support compositions derived from the core multi-layer oral composition of Table 1, wherein functional support module B4 is selected and optionally combined with module B1.

TABLE 2
Exemplary Cardiovascular and Circulatory Support Oral Compositions of the Invention

| | | Exemplary Amount | |
|---|---|---|---|
| Component Category | Ingredient | (per unit dose) | |
| A. Primary Bioactive | Nigella sativa (seed powder, extract, or | 50-300 | mg |
| Modulation Layer | oil; standardized to 0.1-5% | | |
| | thymoquinone) | | |
| | Piper nigrum extract (standardized to 90- | 2-15 | mg |
| | 95% piperine) | | |
| B. Functional Support | | | |
| Modules | | | |
| B4. Cardiovascular/ | Hawthorn berry extract (flavonoids/ | 25-150 | mg |
| Circulatory Module | vitexin) | | |
| | Ginkgo biloba extract (24% flavone | 25-120 | mg |
| | glycosides) | | |
| | Danshen (Salvia miltiorrhiza) extract | 25-150 | mg |
| | L-Citrulline | 50-200 | mg |
| | Nattokinase (≥2000 FU/g) | 25-75 | mg |
| | Magnesium (citrate, glycinate, or | 25-100 | mg |
| | equivalent) | | |
| B1. Anti-Inflammatory/ | Curcuma longa extract (curcuminoids) | 5-100 | mg |
| Antioxidant Module | Ginger root powder or extract | 10-100 | mg |
| (Optional) | Vitamin E (mixed tocopherols) | 5-40 | mg |
| C. Delivery, Stability, and | Rice flour, acacia gum, MCC, or | q.s. | |
| Excipient Layer | combinations thereof | | |
| | Rosemary extract or Vitamin E | 0.1-20 | mg |
| | (natural antioxidants) | | |
| Total Unit Dose | | 400-700 | mg |

Table 3 illustrates exemplary metabolic, glycemic, lipid, and weight-management support compositions derived from the core multi-layer oral composition of Table 1, wherein functional support modules B2 and B1 are selected.

TABLE 3
Exemplary Metabolic Support Oral Compositions

| | | Exemplary | |
|---|---|---|---|
| Component Category | Ingredient | Amount | |
| A. Primary Bioactive Modulation | Nigella sativa (0.1-5% | 50-300 | mg |
| Layer | thymoquinone) | | |
| | Piper nigrum extract (90-95% | 2-15 | mg |
| | piperine) | | |
| B2. Digestive/Metabolic Module | Berberine extract | 50-150 | mg |
| | Cinnamon bark extract | 10-120 | mg |
| | Carom powder or extract | 10-200 | mg |
| | Triphala extract | 20-150 | mg |
| B1. Anti-Inflammatory Module | Curcuma longa extract | 5-100 | mg |
| | Green Tea extract (EGCG) | 10-200 | mg |
| C. Delivery & Stability Layer | Natural excipients and antioxidants | q.s. | |
| Total Unit Dose | | 400-700 | mg |

Table 4 illustrates high-potency immune support compositions derived from the core oral composition of Table 1, employing elevated Nigella sativa and piperine ranges.

TABLE 4
Exemplary High-Potency Immune Compositions

| Component Category | Ingredient | Exemplary Amount | |
|---|---|---|---|
| A. Primary Layer | Nigella sativa (0.1-5% TQ) | 150-400 | mg |
| | Piper nigrum extract | 5-20 | mg |
| B1. Immune/Antioxidant Module | Curcumin, Quercetin, | 5-150 mg each | |
| | Resveratrol | | |
| C. Excipient Layer | Natural binders and antioxidants | q.s. | |
| Total Unit Dose | | 500-800 | mg |

Table 5 illustrates digestive and gastrointestinal support compositions derived from Table 1, wherein Module B2 predominates.

TABLE 5
Exemplary Digestive Support Compositions

| Component Category | Ingredient | Exemplary Amount | |
|---|---|---|---|
| A. Primary Layer | Nigella sativa | 25-200 | mg |
| | Piper nigrum extract | 2-15 | mg |

| | | |
|---|---|---|
| B2. Digestive Module | Carom, Ginger, Fennel, Artichoke | 10-200 mg each |
| C. Delivery Layer | Natural excipients | q.s. |

Table 6 illustrates oral joint and musculoskeletal support compositions derived from Table 1.

TABLE 6
Exemplary Joint Support Compositions

| | | Exemplary | |
|---|---|---|---|
| Component Category | Ingredient | Amount | |
| A. Primary Layer | Nigella sativa | 50-250 | mg |
| | Piper nigrum extract | 2-10 | mg |
| B1. Structural/Anti-Inflammatory | Curcumin, Cissus, | 25-200 | mg |
| Module | Boswellia | | |

| | | |
|---|---|---|
| C. Delivery Layer | Natural excipients | q.s. |

Table 7 illustrates neuro-cognitive and stress support compositions derived from Table 1

TABLE 7
Exemplary Neuro-Support Compositions

| Component Category | Ingredient | Exemplary Amount | |
|---|---|---|---|
| A. Primary Layer | Nigella sativa | 50-200 | mg |
| | Piper nigrum extract | 2-10 | mg |
| B3. Neuro-Endocrine Module | Ashwagandha, Bacopa, Lion's | 25-200 | mg |
| | Mane | | |

| | | |
|---|---|---|
| C. Delivery Layer | Natural excipients | q.s. |

Table 8 illustrates respiratory and allergy support compositions derived from Table 1.

TABLE 8
Exemplary Respiratory Support Compositions

| Component Category | Ingredient | Exemplary Amount | |
|---|---|---|---|
| A. Primary Layer | Nigella sativa | 50-250 | mg |
| | Piper nigrum extract | 2-15 | mg |
| B1. Respiratory Module | Turmeric, Licorice, Holy Basil | 25-150 | mg |

| | | |
|---|---|---|
| C. Delivery Layer | Natural excipients | q.s. |

Table 9 illustrates renal and detoxification support compositions derived from Table 1.

TABLE 9
Exemplary Detox Support Compositions

| Component | | Exemplary |
|---|---|---|
| Category | Ingredient | Amount |
| A. Primary Layer | Nigella sativa | 50-200 mg |
| B2. Detox Module | Dandelion, Milk | 25-200 mg |
| | Thistle, Triphala | |
| C. Delivery Layer | Natural excipients | q.s. |

Table 10 illustrates men's health and prostate support compositions derived from Table 1.

TABLE 10
Exemplary Men's Health Compositions

| Component Category | Ingredient | Exemplary Amount |
|---|---|---|
| A. Primary Layer | Nigella sativa | 50-250 mg |
| B5. Hormonal Module | Saw Palmetto, | 25-150 mg |
| | Pygeum | |
| C. Delivery Layer | Natural excipients | q.s. |

Table 11 illustrates women's hormonal & fertility support compositions derived from Table 1.

TABLE 11
Exemplary Women's Health Compositions

| Component Category | Ingredient | Exemplary Amount |
|---|---|---|
| A. Primary Layer | Nigella sativa | 50-200 mg |
| B5. Hormonal Module | Shatavari, Vitex, Maca | 25-150 mg |
| C. Delivery Layer | Natural excipients | q.s. |

Table 12 illustrates lower-dose pediatric oral compositions derived from Table 1.

TABLE 12
Exemplary Pediatric Syrup Compositions

| Component | | Exemplary |
|---|---|---|
| Category | Ingredient | Amount |
| A. Primary Layer | Nigella sativa | 5-50 mg |
| B2/B1 Modules | Digestive or | 5-50 mg |
| | immune botanicals | |
| Liquid Base | Honey, water, glycerin | q.s. |

Table 13 illustrates exemplary topical lipid-based compositions derived from the core system of Table 1 and configured to support skin healing, barrier repair, and sensitive or compromised skin conditions.

TABLE 13
Exemplary Skin Healing & Sensitive Skin Balm Compositions

| | | Exemplary Amount | |
|---|---|---|---|
| Component Category | Ingredient | (per 100 g) | |
| A. Primary Bioactive | Nigella sativa (oil, powder, or extract; | 2-10 | g |
| Modulation Layer | 0.1-5% TQ) | | |
| B. Barrier Repair/Lipid | Shea butter | 10-20 | g |
| Support Module | Jojoba oil | 30-60 | g |
| | Squalane (olive or sugar-derived) | 3-10 | g |
| | Oat lipid oil or Calendula oil | 3-10 | g |
| C. Structural/Consistency | Candelilla wax or beeswax | 8-15 | g |
| Layer | | | |
| D. Soothing/Anti-Irritant | Chamomile, Licorice, Madecassoside | 0.1-3 | g |
| Botanicals | | | |
| E. Stability Layer | Vitamin E, Rosemary extract | 0.1-2 | g |

Table 14 illustrates topical balm compositions derived from Table 1 and configured for musculoskeletal comfort using counter-irritant and anti-inflammatory botanicals

TABLE 14
Exemplary Muscle & Joint Relief Balm Compositions

| Component Category | Ingredient | Exemplary Amount (per 100 g) | |
|---|---|---|---|
| A. Primary Layer | Nigella sativa (0.1-5% TQ) | 2-8 | g |
| B. Anti-Inflammatory | Turmeric, Boswellia, Arnica extracts | 1-5 | g |
| Module | | | |
| C. Counter-Irritant | Menthol, Camphor, Capsicum, | 0.1-6 | g |
| Module | Wintergreen | | |
| D. Lipid Carrier Layer | Jojoba, Tamanu, Castor, Moringa oils | 30-60 | g |
| E. Structural Layer | Shea butter, waxes | 10-25 | g |
| F. Stability Layer | Vitamin E, Rosemary extract | 0.1-2 | g |

Table 15 illustrates aromatic topical ointment compositions derived from Table 1 and configured to support respiratory comfort.

TABLE 15
Exemplary Respiratory Support Ointment Compositions

| Component Category | Ingredient | Exemplary Amount (per 100 g) | |
|---|---|---|---|
| A. Primary Layer | Nigella sativa (oil or extract) | 5-10 | g |
| B. Aromatic Support | Eucalyptus, Peppermint, | 1-6 | g |
| Module | Camphor | | |
| C. Soothing Botanicals | Licorice, Carom oil | 1-5 | g |
| D. Lipid Carrier Layer | Jojoba oil | 60-75 | g |
| E. Structural Layer | Candelilla wax | 8-12 | g |
| F. Stability Layer | Vitamin E, Rosemary extract | 0.1-2 | g |

Table 16 illustrates topical compositions derived from Table 1 and configured for skin tone balancing and microbial control.

TABLE 16
Exemplary Anti-Acne & Pigmentation Compositions

| Component Category | Ingredient | Exemplary Amount (per 100 g) | |
|---|---|---|---|
| A. Primary Layer | Nigella sativa oil or encapsulated | 5-15 | g |
| | TQ | | |
| B. Antimicrobial Module | Tea Tree, Frankincense | 0.5-3 | g |
| C. Tone-Balancing | Niacinamide, Licorice, Arbutin | 3-10 | g |
| Actives | | | |
| D. Lipid Base | Squalane, Rosehip, Tamanu oils | 40-60 | g |
| E. Structural Layer | Shea butter, wax | 5-15 | g |
| F. Stability Layer | Vitamin E, Rosemary extract | 0.1-2 | g |

Table 17 illustrates topical botanical antimicrobial compositions derived from Table 1.

TABLE 17
Exemplary Antifungal Topical Compositions

| Component Category | Ingredient | Exemplary Amount (per 100 g) | |
|---|---|---|---|
| A. Primary Layer | Nigella sativa oil (0.1-5% TQ) | 5-20 | g |
| B. Antifungal Module | Neem, Tea Tree, Lemongrass, Thyme | 1-6 | g |
| C. Barrier Support | Oat lipid oil, Propolis | 1-5 | g |
| D. Lipid Carrier Layer | MCT, Olive, Jojoba oils | 40-60 | g |
| E. Structural Layer | Shea butter, wax | 5-15 | g |
| F. Stability Layer | Vitamin E, Rosemary extract | 0.1-2 | g |

Table 18 illustrates multifunctional topical balms derived from Table 1 and configured for neurosensory modulation and skin rejuvenation.

TABLE 18
Exemplary Anti-Aging & Neuromodulatory Balms

| Component | | Exemplary Amount | |
|---|---|---|---|
| Category | Ingredient | (per 100 g) | |
| A. Primary Layer | Nigella sativa (0.1-5% TQ) | 5-10 | g |
| B. Regenerative | Bakuchiol, CoQ10, | 0.5-5 | g |
| Module | Centella | | |
| C. Sensory | Peppermint, Clove, | 0.5-5 | g |
| Modulation | Ginger | | |
| Module | extracts | | |
| D. Lipid Support | Jojoba, Sea | 40-60 | g |
| | Buckthorn, Squalane | | |
| E. Structural Layer | Shea butter, wax | 10-20 | g |
| F. Stability Layer | Vitamin E, | 0.1-2 | g |
| | Rosemary extract | | |

Table 19 illustrates oil-based compositions derived from Table 1 and configured for scalp and follicular support.

TABLE 19
Exemplary Hair & Scalp Oil Compositions

| | | Exemplary Amount | |
|---|---|---|---|
| Component Category | Ingredient | (per 100 mL) | |
| A. Primary Layer | Nigella sativa oil | 10-30 | mL |
| B. Follicular | Pumpkin seed, Castor, | 20-50 | mL |
| Support Oils | Bhringraj | | |
| C. Growth-Support | EGCG, Caffeine, | 0.1-3% | |
| Actives | Biotin | | |
| D. Sensory Oils | Peppermint, Rosemary | 0.1-1 | mL |
| E. Stability Layer | Vitamin E | 0.1-1 | mL |

Table 20 illustrates rinse-off and leave-on body care compositions derived from Table 1.

TABLE 20
Exemplary Body Lotion & Cleanser Compositions

| Component Category | Ingredient | Exemplary Amount |
|---|---|---|
| A. Primary Layer | Nigella sativa oil or extract | 2-15% |
| B. Lipid/Emollient Phase | Shea butter, Jojoba, Squalane | 5-20% |
| C. Aqueous Phase | Water, Aloe juice | q.s. |
| D. Cleansing System (optional) | Glucosides, SCI | 5-30% |
| E. Botanical Enhancers | Neem, Turmeric, Calendula | 0.1-5% |
| F. Preservation & Stability | Natural preservatives, antioxidants | q.s. |

1. A multifunctional natural bioactive composition comprising:
a Nigella sativa provided in the form selected from the group consisting of a seed powder, an extract, and an oil, wherein said Nigella sativa contains thymoquinone;
a Piper nigrum extract standardized to contain piperine, wherein said Piper nigrum extract is selected from the group consisting of an oleoresin and a fruit-derived fraction;
at least one pharmaceutically, nutraceutically, or cosmetically acceptable excipient;
wherein said piperine is present in an amount effective to enhance systemic bioavailability, topical bioavailability, and/or functional interaction of said thymoquinone; and
further wherein the bioactive composition is formulated for oral or topical administration to provide anti-inflammatory, immunomodulatory, or antioxidant effects.

2. The composition of claim 1, wherein said Nigella sativa is standardized to contain at least 0.1% thymoquinone.

3. The composition of claim 1, wherein said Piper nigrum extract is standardized to contain at least 90% said piperine.

4. The composition of claim 1, further comprising Curcuma longa extract containing curcuminoids.

5. The composition of claim 1, further comprising Carom (Trachyspermum ammi, synonym Carum copticum).

6. The composition of claim 1 further comprising a plant-derived bioactive agent selected from one or more of the group consisting of curcuminoids, carom, Salvia hispanica L., berberine, ginger, cinnamon, fennel, cissus extracts, Boswellia, adaptogenic botanicals, and polyphenols.

7. The composition of claim 1, wherein said composition is formulated in a form selected from the group consisting of a capsule, a tablet, a powder, a syrup, a balm, a cream, an ointment, a gel, an oil, and a lotion.

8. The composition of claim 7, further comprising a lipid carrier selected from the group consisting of a beeswax, a candelilla wax, a shea butter, a jojoba oil, a sunflower oil, an olive oil, and a squalane.

9. The composition of claim 1, wherein said thymoquinone is provided in a form selected from the group consisting of a raw botanical component, a standardized extract, an encapsulated form, a nano-dispersion, and a liposomal form.

10. A multifunctional natural bioactive composition comprising: a Piper nigrum extract standardized to contain piperine; one or more plant-derived bioactive agents selected to provide digestive, musculoskeletal, inflammatory, metabolic, or epithelial support; and at least one pharmaceutical excipient;
wherein said piperine is present in an amount effective to enhance bioavailability, tissue interaction, and synergistic biological activity of said plant-derived bioactive agents;
wherein said plant-derived bioactive agents selected from one or more of the group consisting of curcuminoids, cissus extracts, carom, fennel, chia, and hyaluronic acid; and
further wherein the composition is formulated for oral or topical administration.

11. The composition of claim 10, wherein the composition is formulated in a form selected from the group consisting of a tablet and a capsule.

12. A natural medicinal composition configured as a multi-layer biological modulation system comprising:
a primary biological modulation layer comprising one or more plant-derived bioactive agents;
a secondary functional support layer comprising (i) one or more plant-derived lipid systems; and (ii) one or more functional support agents selected from the group consisting of adaptogenic agents, antioxidants, and structural biomolecules;
a delivery and interaction layer configured to enhance bioavailability, tissue residence time, synergistic interaction, and biological persistence; and
wherein said one or more plant-derived bioactive agents selected from the group consisting of thymoquinone-containing botanicals and piperine-containing botanicals.

13. The composition of claim 12, wherein said primary biological modulation layer comprises said thymoquinone-containing botanicals.

14. The composition of claim 12, further comprising Nigella sativa standardized to contain at least 0.1% thymoquinone.

15. The composition of claim 14, further comprising Piper nigrum extract standardized to contain at least 90% piperine.

16. The composition of claim 15, further comprising Curcuma longa extract containing curcuminoids.

17. The composition of claim 16, further comprising Carom (Trachyspermum ammi, synonym Carum copticum).

18. The composition of claim 17, further comprising Salvia hispanica L.

19. The composition of claim 18, wherein the natural medicinal composition is formulated in a form selected from the group consisting of a capsule, a tablet, a powder, a syrup, a balm, a cream, an ointment, a gel, and an oil.

20. The composition of claim 14, wherein said thymoquinone is provided in a form selected from the group consisting of a raw botanical component, a standardized extract, an encapsulated form, a nano-dispersion, and a liposomal form.
